(19) United States
(12) Patent Application Publication
(10) Pub. No.: US 2018/0240549 A1
(43) Pub. Date: Aug. 23, 2018

(54) Computer-Implemented Apparatus And Method For Predicting Performance Of Surgical Strategies

(71) Applicant: University of Exeter, Exeter, Devon (GB)

(72) Inventors: John Terry, Exeter, Devon (GB); Marc Goodfellow, Exeter, Devon (GB)

(21) Appl. No.: 15/753,372

(22) PCT Filed: Aug. 18, 2016
(86) PCT No.: PCT/GB2016/052556

§ 371 Date: Feb. 19, 2018

(30) Foreign Application Priority Data
Aug. 19, 2015 (GB) 1514740.8

(51) Int. Cl. G16H 50/20; G16H 50/50; G06N 3/10

(52) U.S. Cl. CPC G16H 50/20; G06N 3/10; G16H 50/50

(57) ABSTRACT

A computer-implemented apparatus for predicting an effect of a proposed surgical strategy for treatment of epilepsy and/or epileptic seizures, configured to generate synthetic brain activity data in at least some of the nodes of a brain network model, and repeatedly: a) simulate or effect a surgical strategy comprising removal of at least one node and/or edge from said brain network and subsequently recalculate said BNI value thereof; and b) calculate a value ΔBNI representative of a magnitude of change in BNI following removal of said at least one node/edge from said brain network, so as to output multiple ΔBNI values, or data representative thereof, corresponding to respective multiple proposed surgical strategies, each comprising removal of respective nodes/edges or sets of nodes/edges from said brain network.

This invention relates generally to a computer-implemented apparatus and method for predicting performance and/or (potential) effects of a surgical strategy and, more particularly but not necessarily exclusively, to such a computer-implemented apparatus and method for assessing performance and/or (potential) effects of a surgical strategy in respect of epilepsy patients.

Epilepsy is the most common serious neurological disease, affecting 1.2% of the population in the UK. Epilepsy is often mistakenly viewed as easily treated, whereas, in fact, 40% of new-onset epilepsy remains uncontrolled after one year of antiepileptic drug (AED) treatment, and over 30% of people with epilepsy never respond to treatment, with consequent morbidity and mortality. Epilepsy is directly responsible for over 100 deaths per year in the UK, and is the fifth most common cause of avoidable years of life lost in men and eighth in women. It is the leading cause of repeated unplanned admissions to NHS hospitals, and is estimated to cost the EU 15.5 billion euros per annum.

Anti-epileptic drugs (AEDs) are usually the first choice of treatment for epilepsy. However, only about 70% of people with the condition are able to control their seizures with AEDs. For some people, brain surgery may be an option. However, this is only the case if removing the area of the brain where epileptic activity starts would not cause damage or disability. If successful, there is a chance epilepsy in the patient will be cured. On the other hand, although surgery is a valuable option for pharmacologically intractable cases of epilepsy, success rates are far from optimal, For example, sustained positive outcome 12 months post-surgery is currently only achieved in around half of patients, and can be as low as 15% in extra-temporal cases. The reasons why surgery can succeed or fail often remain unclear due to a fundamental lack of understanding of the seizure generating (ictogenic) capabilities of the brain, and the inability to assess or predict the consequences of surgical resections. A challenge in identifying the cause of surgery failure lies in the potential involvement of both immediate and longer-term mechanisms. For example, cases with recurrence of seizures in the short-term are most likely to remain intractable, and are presumed to be the result of an inadequate surgical resection, such that the seizure generating (ictogenic) mechanisms that existed before surgery still remain. Long-term failure, however, can occur for cases in which the original ictogenic mechanisms were abolished, but new mechanisms emerged post-surgery. Furthermore, some patients may be seizure-free postoperatively, but seizures can emerge when drugs are withdrawn. It is therefore an object of aspects of the present invention to address the problem in epilepsy management of the optimisation of surgery with a view to achieving maximal benefit to the patient.

A prevalent theoretical concept underpinning the surgical treatment of epilepsy is that of the epileptogenic zone, i.e. “an area of the cortex that is indispensable for the generation of epileptic seizures” (Rosenow, F. and H. Lüders 2001. Presurgical evaluation of epilepsy. Brain, Pp. 1683-1700.). The goal of epilepsy surgery in this paradigm is the removal or disconnection of the epileptogenic zone, since in a “localised” view of epilepsy, such perturbations would render a patient seizure free. Unfortunately, this is a retrospective definition; the epileptogenic zone can only be identified in patients for whom surgery was successful and is not, therefore, a theory that aids the prediction of an optimal surgical strategy. Rather, a combination of observations is acquired during pre-surgical planning regarding aspects of the epileptic brain that are presumed to correlate to the epileptogenic zone. These include, for example, regions of the brain that are first to produce pathologic electrographic activity when seizures occur and regions that display evidence of abnormal structure that perhaps contribute to ictogenicity. Concordance of these features is presumed to indicate a clear epileptogenic zone and in these cases surgery is most likely to succeed. However, non-concordance of these features often occurs and, even in cases for which well-defined focal epileptogenic zones caused by specific lesions are assumed (for example, mesial temporal lobe epilepsy), surgical resection can be unsuccessful.

Thus, it would be highly desirable to provide an apparatus and method that addresses at least some of these issues and provides an improved facility for assessing and/or predicting the effects of a proposed surgical intervention in respect of epilepsy patients.

In accordance with an aspect of the present invention, there is provided a computer-implemented apparatus for predicting an effect of a proposed surgical strategy for treatment of epilepsy and/or epileptic seizures, the apparatus comprising:
a device configured to generate a brain network model representative of brain data, wherein nodes in the network model correspond to brain regions of said brain data and connections between the nodes of the network model correspond to structural and/or functional connections between the brain regions;
a device configured to generate synthetic brain activity data in at least some of the nodes of the network model;
a device configured to compute a representative brain network ictogenicity (BNI) value from the synthetic brain activity data, wherein said BNI value is representative of a probability of said brain network to generate seizures;
a device configured to, repeatedly: a) simulate or effect a surgical strategy comprising removal of at least one node and/or edge from said brain network and subsequently recalculate said BNI value thereof; and b) calculate a value ΔBNI representative of a magnitude of change in BNI following removal of said at least one node/edge from said brain network, wherein said ΔBNI value is indicative of an effectiveness of removal of said at least one node/edge from said brain network in reducing said probability of said brain network to generate seizures; thereby to output multiple ΔBNI values, or data representative thereof, corresponding to respective multiple proposed surgical strategies, each comprising removal of respective nodes/edges or sets of nodes/edges from said brain network.

In prior art systems, there is required an a priori assumption or hypothesis of the nature of the brain network and a link between abnormal regions of the brain and the generation of seizures. In contrast, the present invention does not require this a priori assumption or hypothesis of nodes that form a seizure focus to define a seizure onset zone. Instead, the present invention provides a system and method for systematic exploration and quantification of the effect of removing any node on seizure generation, without any a priori assumptions or hypotheses being made as to the pathology of the brain. In an exemplary embodiment of the present invention, the apparatus further comprises a device configured to receive patient brain data, wherein said brain network model is generated from said patient brain data. In an exemplary embodiment, the apparatus comprises a device for selecting, from said multiple proposed surgical strategies, a proposed surgical strategy based on the ΔBNI value calculated in respect thereof. For example, the proposed surgical strategy having the highest ΔBNI value calculated in respect thereof may be selected and, optionally, data representative thereof, for example in the form of which node(s)/edge(s) should be removed, may be output.

Optionally, the synthetic brain activity data comprises, or is configured to simulate, recurrent seizures within said brain network model. In an exemplary embodiment, the apparatus may be configured to place the brain network model close to a transition between brain states. Optionally, one or more parameters of said brain network model may be configured model to place the model close to a saddle-node on invariant circle (SNIC) bifurcation, but the present invention is not necessarily intended to be limited in this regard and other methods of placing the brain network model close to transition between brain states will be apparent to a person skilled in the art.

In an exemplary embodiment, said BNI value is calculated by:

$$B N I = \frac{Total time spent in seizures}{Duration of reference time period}$$

The ΔBNI value, in respect of a brain network having node i removed, may be calculated using the equation:

$$Δ {BNI}^{i} = \frac{{BNI}_{pre}^{i} - {BNI}_{post}^{i}}{{BNI}_{pre}^{i}}$$

wherein BNIⁱₚᵣₑ is the BNI value of the brain network before removal of node i and BNIⁱₚₒₛₜ is the BNI value of the brain network after removal of the node i.

Apparatus according to an exemplary embodiment of the invention comprises a device configured to identify, from said synthetic brain activity data, nodes having contiguous seizure epochs, calculate the BNI value for each of said identified nodes, and select the highest BNI value thus calculated as a representative BNI for said brain network.

In accordance with another aspect of the present invention, there is provided a computer-implemented method for predicting an effect of a proposed surgical strategy for treatment of epilepsy and/or epileptic seizures, the method comprising:
generating a brain network model representative of brain data, wherein nodes in the network model correspond to brain regions of said brain data and connections between the nodes of the network model correspond to structural and/or functional connections between the brain regions;
generating synthetic brain activity data in at least some of the nodes of the network model;
computing a representative brain network ictogenicity (BNI) value from the synthetic brain activity data, wherein said BNI value is representative of a probability of said brain network to generate seizures;
repeatedly:
a) simulating or effecting a proposed surgical strategy comprising removal of at least one node and/or edge from said brain network and subsequently recalculate said BNI value thereof; and
b) calculating a value ΔBNI representative of a magnitude of change in BNI following removal of said at least one node/edge from said brain network, wherein said ΔBNI value is indicative of an effectiveness of removal of said at least one node/edge from said brain network in reducing said probability of said brain network to generate seizures; thereby to output multiple ΔBNI values, or data representative thereof, corresponding to respective multiple proposed surgical strategies, each comprising removal of respective nodes/edges or sets of nodes/edges from said brain network. In an exemplary embodiment, the method may comprise selecting, from said multiple proposed surgical strategies, a proposed surgical strategy based on the ΔBNI value calculated in respect thereof. For example, the proposed surgical strategy having the highest ΔBNI value calculated in respect thereof may be selected and, optionally, data representative thereof, for example in the form of which node(s)/edge(s) should be removed, may be output.

These and other aspects of the present invention will be apparent from the following specific description, in which embodiments of the present invention are described, by way of examples only, and with reference to the accompanying drawings, in which:

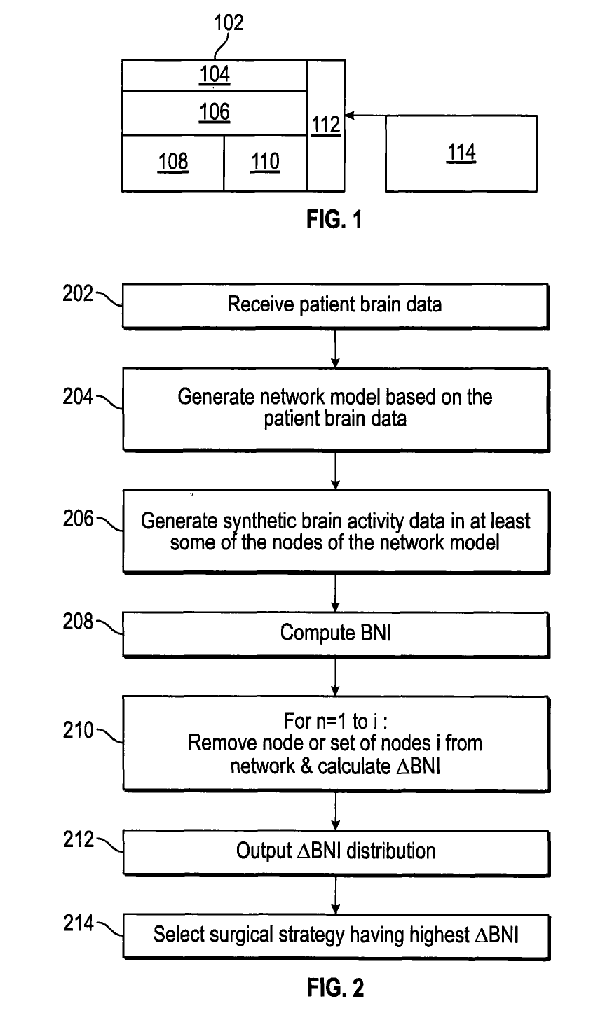

FIG. 1 is a schematic block diagram illustrating apparatus according to an exemplary embodiment of the present invention;

FIG. 2 is a schematic diagram illustrating steps performed by apparatus according to an exemplary embodiment of the present invention;

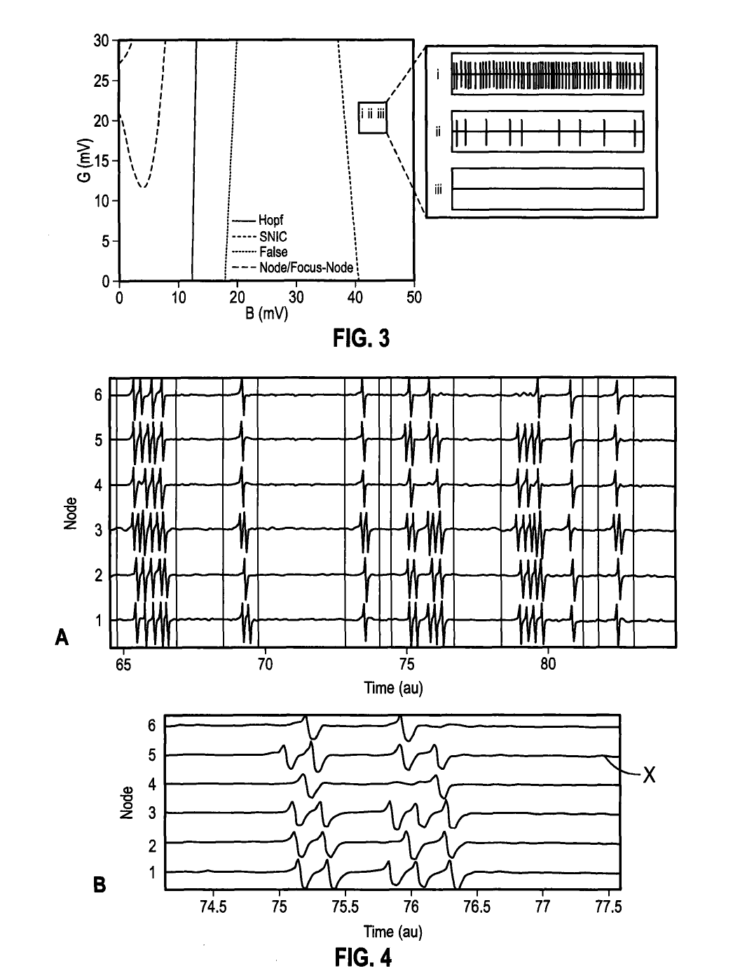

FIG. 3 illustrates bifurcations in the model used by the apparatus of FIG. 1 and examples of spiking due to noise: the left panel shows bifurcations in the model of a single node over parameters B and G and three parameter choices are indicated by roman numerals (1) B=41, (ii) B=42, (iii) B=44; simulations of each of the parameter choices are shown in the right-hand panel, indicating an increased excitability as the model moves closer to the SNIC bifurcation; and

FIG. 4 illustrates the concept of extraction of seizure epochs and seizure onset zones; A: example simulation in a six-node network, generating short epochs of spiking dynamics (seizures), B: example of seizure onset zone in an example seizure epoch, wherein the seizure onset node is indicated by ‘X’.

Although surgery is a valuable option for pharmacologically intractable cases of epilepsy, success rates are far from optimal, and the reasons why surgery can succeed or fail have remained unclear, which stems from a fundamental lack of understanding if the seizure generating (ictogenic) capabilities of the brain, and the consequences of surgical resections. The inventors have determined, through extensive innovative effort and novel methods and systems, that seizures are an emergent property of a network within which the existence of pathological zones is neither a necessary nor sufficient condition for seizures to emerge. The novel method and apparatus described hereinafter can be used to demonstrate that typically presumed correlates of the region of tissue to resect, such as the location of pathological nodes or seizure onset zones, do not necessarily predict the best resection strategy. Thus, exemplary embodiments of the present invention provide a framework for assessing, predicting and/or optimising the outcome of surgical strategies in epilepsy patients.

FIG. 1 shows a computing device 102 including a processor 104 and memory 106. The memory includes patient brain data 108 and an application 110 for processing the patient brain data. The computing device further includes an interface 112 for communicating with remote devices. The computing device can also have other conventional features, such as display, user-interface, etc. that need not be described herein in detail.

In this exemplary system, the interface 112 of the computing device is connected to a brain data recording machine 114, which may be an EEG machine, MEG machine, MRI scanner, or the like. In the example, the application 110 is configured to receive data 108 from the machine 114 (via any wired or wireless data transfer medium) and process it as described below. In other embodiments, the brain data 108 may be received by the computing device in another manner, e.g. over a communications network or from transportable storage medium, such as a DVD. It will be further understood that in alternative embodiments, at least some of the steps of the application 110 may be performed sequentially, or in parallel on one or more remote processing devices.

FIG. 2 illustrates schematically the steps of a method according to an exemplary embodiment of the present invention. The skilled person will understand that the steps can be coded using any suitable programming language and/or data structures. It will also be understood that in alternative embodiments some of the steps may be omitted and/or re-ordered and/or additional steps may be performed.

At step 202, the application 110 receives patient brain data from a source as described above. At step 204, the application generates a network model based on the patient brain data. Network analysis has been proposed, generally, in the study of brain function and, more particularly, in the study of epilepsy, and offers a framework to characterise the organisation of brain networks. Network analysis reduces complex systems such as the brain to a collection of “nodes” and “edges”. Nodes represent functional or structural elements of the network, in this case the brain regions of the patient brain data, and edges are any type of relation between brain regions, representing either a structural or functional connection between brain regions, as recorded in the source patient brain data. Together, these two building blocks enable characterisation of the organisation of brain networks.

The structure of the above-mentioned network model may be inferred from the patient brain data using any one of a number of methods, as set out, for example, in Eric van Diessen, Sander J. H. Diederen, Kees P. J. Braun, Floor E. Jansen and Cornelis J. Stam, Functional and structural brain networks in epilepsy: What have we learned?. Epilepsia, 54(11): 1855-1865, 2013 (the contents of which are hereby incorporated by reference), Benjamin, O., Fitzgerald, T. H. B., Ashwin, P. et al, A phenomenological model of seizure initiation suggests network structure may explain seizure frequency in idiopathic generalised epilepsy. J Math Neurosci 2012, Vol. 2, No. 1, pp. 1-41 (the contents of which are hereby incorporated by reference) or alternatively using another measure of nonlinear correlation. Alternatively, a method for inferring brain networks from data, that involves learning parameters of a specific model from such data, may be used, as set out in, for example, Freestone, D. R., Aram, P., Dewar, M., Scerri, K., Grayden, D. B., & Kadirkamanathan, V. (2011). A data-driven framework for neural field modeling. NeuroImage, 56(3), 1043-1058; and Freestone, D. R., Karoly, P. J., Nešić, D., Aram, P., Cook, M. J., & Grayden, D. B. (2014). Estimation of effective connectivity via data-driven neural modeling. Frontiers in Neuroscience, 8 (November), 383.

However, in general and as stated above, the model comprises a set of nodes that correspond to brain regions of the patient brain data and connections between the nodes of the network data structure correspond to connections between the brain regions, as recorded in the source brain data. In the case of EEG data, for instance, the brain regions will correspond to regions of the brain close to the electrodes of the EEG machine.

At step 206, the apparatus generates synthetic brain activity data based on at least some of the nodes of the network model. This can be a dynamic computational model based on features of human seizure data, e.g. one that phenomenologically or physiologically models transitions from interictal to ictal states in brain regions over time. In alternative embodiments, the synthetic brain activity data can be generated using a probabilistic model, for example, representation by a Markov process. Mathematically, seizures in the above-described model can arise due to several different mechanisms, and apparatus according to an exemplary embodiment of the present invention may be configured to place the brain network model close to a transition between brain states. In an exemplary, non-limiting embodiment, parameterisation of the model may, for example, be designed to place the model close to a saddle-node on invariant circle (SNIC) bifurcation, as discussed in Blenkinsop, A. Valentin, A., Richardson, M. P., and Terry, J. R. The dynamic evolution of focal-onset epilepsies-combining theoretical and clinical observations. The European Journal of neuroscience, 25(3):756-770. In this regime, a model node can generate spiking due to noise and this can propagate through networks. This model therefore has the property that it can generate recurrent seizure events, and therefore is a model for both seizure generation as well as epilepsy, and it can be shown that the proximity of the model to the above-mentioned bifurcation determines its degree of “excitability” in the sense of how likely it is to generate spikes. However, other suitable methods for placing the network model close to a transition between brain states will be apparent to a person skilled in the art.

Next, at step 208, the apparatus computes the probability of a brain network to generate seizures, hereinafter referred to as Brain Network Ictogenicity (BNI) and introduced in Chowdbury et al, 2014. A practical approach to quantifying this is by summing the total time spent in seizure compared to a reference time period (seizure days per year, for example):

$$B N I = \frac{Total time spent in seizures}{Duration of reference time period}$$

Thus, using the above-described model, seizure epochs are defined by the presence of spiking activity in the model, which is identifiable by the amplitude of the model output in comparison to periods of time where non-spiking dynamics occur, as illustrated in FIG. 3. Spikes are extracted for each node by applying a threshold to the average absolute amplitude of the model output over a sliding window of length 0.05 s. In order to identify contiguous epochs of spiking activity, a window of length 1 second (which incorporates approximately 4 spikes) is placed around the centre of each spike, for each node. Epochs are considered contiguous, for each node, wherever the end of the window around a spike occurs after the start of the window of the subsequent spike. Once contiguous epochs of spiking have been identified, BNI is calculated for each node (according to the above equation), and the node with the largest BNI is considered representative for a network. Alternative methods of selecting the representative BNI for a network may be considered. However, the above-mentioned method eliminates the need to introduce an arbitrary number of channels in which spiking is required to define a seizure. An example of the extraction of seizure events is shown in FIG. 4A of the drawings.

It will be appreciated that, whilst understanding seizure generation as an emergent property of a network naturally introduces contributions from both nodes (brain regions) and edges (connections between them), from the perspective of surgery, it would not necessarily be intended to just alter edges. Rather, and possibly more typically, the aim would be to resect nodes. Thus, the present invention seeks to assess and/or predict the (potential) effect of surgery based on the contribution of each node to the ictogenicity of the network. More specifically, aspects of the present invention seek to quantify the effect of surgery by measuring the magnitude of the change in BNI that would be observed following the removal of a specific node and/or edge or, more likely, sets of nodes and/or edges. It is this change in BNI, termed herein ΔBNI, that quantifies the contribution to the overall ictogenicity made by a node. Specifically, referring back to FIG. 2 and at step 210, the apparatus defines ΔBNI for one or more given nodes or sets of nodes i (and/or edges) by comparing the BNI before and after its/their removal as follows:

$$Δ {BNI}^{i} = \frac{{BNI}_{pre}^{i} - {BNI}_{post}^{i}}{{BNI}_{pre}^{i}}$$

Given the choice of intrinsic parameters (chosen to place the model close to the above-mentioned SNIC bifurcation) and connectivity structure, free parameters that remain are the global scaling of connectivity strength (α) and the variance of white noise added to each node (σ). We fix σ=650, which is considered sufficient to generate recurrent seizures. Having fixed the noise amplitude, FIG. 1 demonstrates that BNI is dependent upon the choice of global connectivity strength (α). Therefore, a reference value of α is defined for each network, which is the value such that the network spends approximately 50% of its time in spiking dynamics (i.e. BNI≈0.5). ΔBNI is then calculated by comparison to this state, according to the above equation as follows: the model is simulated for 5000 time points for a given noise vector, and BNI is calculated (BNIₚᵣₑ in eq. 1). For each node/edge (or set thereof), i, the node/edge (or set thereof) is removed, the simulation repeated, and BNIₚₒₛₜ calculated. For each network, ΔBNI is calculated for each node, therefore providing a distribution of values of ΔBNI at step 212.

In practice, ΔBNI is equivalent to a quantification of whether resection of a specific brain region (i.e. node(s)/edge(s) i) will sufficiently control seizures and, by estimating this quantity using patient brain data, optimal resections can be planned. Thus, at step 212, the apparatus outputs quantitative values corresponding to the predicted effect (i.e. change) on brain network ictogenicity of resection of one or more respective brain regions, thereby enabling a quantitative evaluation to be made of a number of different surgical strategies and optimal strategy selected. Networks containing nodes with ΔBNI values close to 1 would indicate good prognoses, whereas networks with a lower ΔBNI would be less likely to be cured by a localised resection, and more elaborate surgical strategies might, potentially, be considered. In most cases, the surgical strategy having the highest ΔBNI should be selected. In an exemplary embodiment, at step 214, the apparatus selects a surgical strategy having a highest ΔBNI and outputs data representative thereof, possibly in the form of data indicative of the node(s)/edge(s) that should be removed in accordance with the selected surgical strategy.

Thus, having established a model derived from patient brain data and placed in a state of recurrent seizures, the inventors have developed an apparatus and method for assessing the relative contribution of each node to the overall ictogenicity of a network. The classical approach to surgery assumes that an epileptogenic zone exists, and thus removal of this zone would render a patient seizure free. However, the inventors have determined, through extensive innovative effort and novel methods and systems, that recurrent seizures can arise in networks that do not possess an intrinsically pathological node (i.e. the underlying properties of each region is the same). In this case, the distribution of ΔBNI across the nodes of a network with identical nodes can be heterogeneous. This means that the removal of some nodes can cause a greater reduction in BNI than others, despite all of the nodes having the same intrinsic properties.

The inventors have also determined that the existence of pathological nodes within the network can alter the BNI profile, depending on where in the network the pathology resides. The framework proposed by exemplary embodiments of the present invention can be used to show this using the concept of “seizure onset zones”. For each seizure of a simulation, the “seizure onset zone” is defined as the first node to display a spike at the start of the seizure, as indicated in FIG. 4B. This can be calculated by defining a 0.5 s interval around the start of the seizure and calculating all peaks in the original data for each node in this interval. The node with the earliest peak in this interval may be identified as the seizure onset zone for that particular seizure. If a pathological node were to be placed at each location of a network in turn, and the ΔBNI and seizure onset zones subsequently calculated, the BNI distribution and the network location of the seizure onset zone can be shown to be different, depending on where the pathological node is located, thus demonstrating that a particular network structure can give rise to different optimal targets for resection, depending upon where in the network a pathological region exists, and the method described above solves the problems associated with traditional methods by being able to account for this. Other anomalies in BNI distribution based on the connectivity structure of the model and the interplay between a pathological node and other nodes in the network are also accounted for in the method according to the above-described exemplary embodiment of the present invention, thereby enabling the determination of whether removal of that node can render the network seizure free.

Thus, the present invention moves beyond the traditional qualitative approach and instead quantifies the contribution of each node to ictogenicity by calculating its ΔBNI, i.e. the relative change in ictogenicity brought about by removing one or more nodes/edges from the network, thus enabling one or more potential surgical strategies to be assessed, and the outcome thereof to be predicted, so as to optimise the probability of success of any surgical resection strategy undertaken.

It will be appreciated by a person skilled in the art, from the foregoing description, that modifications and variations can be made to the described embodiments without departing from the scope of the invention as defined by the appended claims.

1. A computer-implemented apparatus for predicting an effect of a proposed surgical strategy for treatment of epilepsy and/or epileptic seizures, the apparatus comprising:
a device configured to generate a brain network model representative of brain data, wherein nodes in the network model correspond to brain regions of said brain data and connections between the nodes of the network model correspond to structural and/or functional connections between the brain regions;
a device configured to generate synthetic brain activity data in at least some of the nodes of the network model;
a device configured to compute a representative brain network ictogenicity (BNI) value from the synthetic brain activity data, wherein said BNI value is representative of a probability of said brain network to generate seizures;
a device configured to, repeatedly: a) simulate or effect a surgical strategy comprising removal of at least one node and/or edge from said brain network and subsequently recalculate said BNI value thereof; and b) calculate a value ΔBNI representative of a magnitude of change in BNI following removal of said at least one node/edge from said brain network, wherein said ΔBNI value is indicative of an effectiveness of removal of said at least one node/edge from said brain network in reducing said probability of said brain network to generate seizures;
thereby to output multiple ΔBNI values, or data representative thereof, corresponding to respective multiple proposed surgical strategies, each comprising removal of respective nodes/edges or sets of nodes/edges from said brain network.

2. Apparatus according to claim 1, further comprising a device configured to receive patient brain data, wherein said brain network model is generated from said patient brain data.

3. Apparatus according to claim 1, comprising a device for selecting, from said multiple proposed surgical strategies, a proposed surgical strategy based on the ΔBNI value calculated in respect thereof.

4. Apparatus according to claim 3, wherein the proposed surgical strategy having the highest ΔBNI value calculated in respect thereof is selected.

5. Apparatus according to claim 4, wherein data representative the selected proposed surgical strategy is output.

6. Apparatus according to claim 5, wherein said data is output in the form of which node(s)/edge(s) should be removed.

7. Apparatus according to claim 1, wherein the synthetic brain activity data comprises, or is configured to simulate, recurrent seizures within said brain network model.

8. Apparatus according to claim 1, configured to place the brain network model close to a transition between brain states.

9. Apparatus according to claim 8, wherein one or more parameters of said brain network model is/are configured model to place the model close to a saddle-node on invariant circle (SNIC) bifurcation

10. Apparatus according to claim 1, wherein said BNI value is calculated by:
$$B N I = \frac{Total time spent in seizures}{Duration of reference time period}$$
The ΔBNI value, in respect of a brain network having node i removed, may be calculated using the equation:
$$Δ {BNI}^{i} = \frac{{BNI}_{pre}^{i} - {BNI}_{post}^{i}}{{BNI}_{pre}^{i}}$$
wherein BNIⁱₚᵣₑ is the BNI value of the brain network before removal of node i and BNIⁱₚₒₛₜ is the BNI value of the brain network after removal of the node i.

11. Apparatus according to claim 1, comprising a device configured to identify, from said synthetic brain activity data, nodes having contiguous seizure epochs, calculate the BNI value for each of said identified nodes, and select the highest BNI value thus calculated as a representative BNI for said brain network.

12. A computer-implemented method for predicting an effect of a proposed surgical strategy for treatment of epilepsy and/or epileptic seizures, the method comprising:
generating a brain network model representative of brain data, wherein nodes in the network model correspond to brain regions of said brain data and connections between the nodes of the network model correspond to structural and/or functional connections between the brain regions;
generating synthetic brain activity data in at least some of the nodes of the network model;
computing a representative brain network ictogenicity (BNI) value from the synthetic brain activity data, wherein said BNI value is representative of a probability of said brain network to generate seizures;
repeatedly:
a) simulating or effecting a proposed surgical strategy comprising removal of at least one node and/or edge from said brain network and subsequently recalculating said BNI value thereof; and
b) calculating a value ΔBNI representative of a magnitude of change in BNI following removal of said at least one node/edge from said brain network, wherein said ΔBNI value is indicative of an effectiveness of removal of said at least one node/edge from said brain network in reducing said probability of said brain network to generate seizures; thereby to output multiple ΔBNI values, or data representative thereof, corresponding to respective multiple proposed surgical strategies, each comprising removal of respective nodes/edges or sets of nodes/edges from said brain network.

13. A method according to claim 12, comprising selecting, from said multiple proposed surgical strategies, a proposed surgical strategy based on the ΔBNI value calculated in respect thereof.

14. A method according to claim 13, wherein the proposed surgical strategy having the highest ΔBNI value calculated in respect thereof is selected.
